(19) United States
(12) Patent Application Publication
(10) Pub. No.: US 2025/0248826 A1
(43) Pub. Date: Aug. 7, 2025

(54) Patellofemoral Component Tracking

(71) Applicant: Howmedica Osteonics Corp., Mahwah, NJ (US)

(72) Inventors: Emily Hampp, Far Hills, NJ (US); Vivienne Clayton, Montville, NJ (US)

(21) Appl. No.: 19/040,409

(22) Filed: Jan. 29, 2025

Related U.S. Application Data
(60) Provisional application No. 63/548,592, filed on Feb. 1, 2024.

(51) Int. Cl. A61F 2/46

(52) U.S. Cl. CPC A61F 2/4657; A61F 2002/4666; A61F 2002/4668

(57) ABSTRACT

Disclosed herein are surgical systems and methods for patellofemoral tracking and patellofemoral joint balancing using the same. The method can include attaching a marker to the patella, monitoring its movement during knee flexion-extension to generate a kinematic profile, and using this data to resect the patella and select a patellar trial. The patellar trial can be analyzed in the same fashion, creating a second profile for selecting the final patellar implant by comparing pre- and post-resection kinematics.

### CROSS-REFERENCE TO RELATED APPLICATIONS

This application claims the benefit of the filing date of U.S. Provisional Patent Application No. 63/548,592 filed on Feb. 1, 2024, the disclosure of which is hereby incorporated herein by reference in its entirety.

### FIELD OF INVENTION

The present invention relates to a surgical system for bone tracking and a method for joint balancing utilizing the same, and more particularly to a surgical system for patellofemoral tracking and a method for patellofemoral joint balancing using the surgical system.

### BACKGROUND OF THE INVENTION

The precise sizing, placement, and balancing of implants for joint bones, such as the patella and femur, are crucial for maintaining the natural kinematics of a patellofemoral joint. Ensuring the biomechanics of this joint remain as close to their natural state as possible can be essential for effective rehabilitation following implantation. For instance, a patellar implant must be accurately sized to fit the resected patella to preserve the natural kinematics during the knee's flexion and extension. The kinematics of the patellofemoral joint post-surgery should ideally mirror the preoperative or pre-disease state of the patient. Ill-fitting patellar implants can disrupt these kinematics, leading to complications like patellar maltracking or other complications.

The selection process for a patellar implant should not be based solely on a single position or a few positions of knee flexion-extension. This is because the patella's movement encompasses six degrees of freedom throughout the entire flexion-extension cycle of the knee joint. Therefore, selecting implants based on limited positions may prove inadequate for ensuring balanced patellofemoral kinematics. Anatomically-shaped patellar implants often aid in better rehabilitation, but their complex geometry makes proper sizing for maintaining kinematic balance a significant challenge.

Implantation of a patellar implant that leads to a patella dimensionally exceeding the natural patella—termed patellar overstuffing—can restrict passive knee flexion motions and disrupt normal patellar kinematic patterns during knee bending. In contrast, patellar understuffing refers to the placement of a patellar implant that leaves the patella smaller compared to its natural size, potentially requiring additional force from the quadriceps during the flexion and extension of the patella and increasing the contact stress upon the patellar surface. Accordingly, it is crucial to employ a patellar implant of proper size to preserve the intended patellofemoral kinematics, avoiding the consequences that come with improper patellar implant selection. Similarly, a surgeon must ensure that the implanted patellofemoral components preserve nature kinematics of the patient's native knee.

Therefore, there exists a need for a surgical system for patellofemoral tracking and a method for patellofemoral joint balancing using the surgical system.

### BRIEF SUMMARY OF THE INVENTION

Disclosed herein are surgical systems and methods for patellofemoral tracking and patellofemoral joint balancing using the same. The method may include attaching a marker to the patella, monitoring its movement during knee flexion-extension to generate a kinematic profile, and using this data to resect the patella and select a patellar trial. The trial patellar implant may be analyzed in the same fashion, creating a second profile for selecting the final patellar implant by comparing pre- and post-resection kinematics. A surgical system centers for tracking patellar motion for patellofemoral alignment can include markers attached to the patella to record its movement across different knee positions, forming a kinematic profile to guide patellar resection. Following resection, the system can evaluate a trial patellar implant through similar tracking, generating another profile. These profiles are compared to determine the best patellar implant and placement for optimal patellofemoral joint alignment.

In accordance with an aspect of the present disclosure, a method for patellofemoral joint balancing is provided. A method according to this aspect may include the steps of positioning a first marker on an unresected patella, tracking the first marker during flexion and extension motion of a knee to track translation and rotation of the unresected patella during the flexion and extension motion, generating a first patellofemoral joint profile from the translation and rotation of the unresected patella, resecting the patella based on the first patellofemoral joint profile, selecting and placing a patellar trial on the resected patella based on the first patellofemoral joint profile. The method may further include the steps of tracking a second marker on the patellar trial or the resected patella during flexion and extension motion of the knee to track translation and rotation of the resected patella and the patellar trial during the flexion and extension motion, generating a second patellofemoral joint profile from the translation and rotation of the resected patella and the patellar trial, and selecting a patellar implant based on comparing the first and second patellofemoral joint profiles.

Continuing in accordance with this aspect, the method may include a step of selecting and placing a second patellar trial if a difference between the first and second patellofemoral joint profiles exceeds a predetermined threshold.

Continuing in accordance with this aspect, the step of selecting the patellar implant may include selecting a patellar implant that is similar to the patellar trial if a difference between the first and second patellofemoral joint profiles is within a predetermined threshold.

Continuing in accordance with this aspect, the method may further include a step of tracking a third marker on the patellar implant or the resected patella during flexion and extension of the knee to track translation and rotation of the resected patella and the patellar implant during the flexion and extension motion and generating a third patellofemoral joint profile from the translation and rotation of the resected patella and the patellar implant. The method may further include a step of comparing the first and third patellofemoral joint profiles. The method may further include a step of selecting and placing a second patellar implant if a difference between the first and third patellofemoral joint profiles exceeds a predetermined threshold.

Continuing in accordance with this aspect, the method may include placing a first tracker on a femur and a second tracker on a tibia. The first and second trackers may include detectors to detect a position of the first and second markers.

Continuing in accordance with this aspect, the step of tracking the first marker may include tracking anterior-posterior translation, medial-lateral translation, and super-inferior translation of the unresected patella. The step of tracking the first marker may include tracking medial rotation, lateral rotation, superior rotation, inferior rotation, and axial rotation of the unresected patella.

Continuing in accordance with this aspect, the step of tracking the second marker may include tracking anterior-posterior translation, medial-lateral translation, and super-inferior translation of the resected patella or the patellar trial. The step of tracking the second marker may include tracking medial rotation, lateral rotation, superior rotation, inferior rotation and axial rotation of the resected patella or the patellar trial.

Continuing in accordance with this aspect, the step of positioning a first marker may include placing at least one magnet in contact with the unresected patella.

In accordance with another aspect of the present disclosure, a method for patellofemoral joint balancing is provided. A method according to this aspect may include the steps of positioning a first sensor on an unresected patella, tracking the first sensor during flexion and extension motion of a knee to track the unresected patella during the flexion and extension motion, generating a first patellofemoral joint profile from the tracked unresected patella, resecting the patella based on the first patellofemoral joint profile, selecting and placing a patellar trial on the resected patella based on the first patellofemoral joint profile, tracking a second sensor on the patellar trial during flexion and extension motion of the knee to track the patellar trial during the flexion and extension motion, generating a second patellofemoral joint profile from the tracked patellar trial, and selecting a patellar implant based on comparing the first and second patellofemoral joint profiles.

Continuing in accordance with this aspect, the step of tracking the first sensor may include tracking an inertial measurement unit on the unresected patella.

Continuing in accordance with this aspect, the step of tracking the first sensor may include tracking a load sensor on the unresected patella. The step of tracking the first sensor may include tracking translation and rotation of the unresected patella. The step of tracking the first sensor may include tracking load values of the unresected patella. The step of tracking the first sensor may include tracking velocity and acceleration of the unresected patella.

Continuing in accordance with this aspect, the step of tracking the second sensor may include tracking an inertial measurement unit on the patellar trial. The step of tracking the second sensor may include tracking a load sensor on the unresected patella. The step of tracking the second sensor may include tracking translation and rotation of the patellar trial. The step of tracking the second sensor may include tracking load values of the patellar trial. The step of tracking the second sensor may include tracking velocity and acceleration of the patellar trial.

Continuing in accordance with this aspect, the step of selecting the patellar implant may include selecting a patellar implant that is similar to the patellar trial if a difference between the first and second patellofemoral joint profiles is within a predetermined threshold.

Continuing in accordance with this aspect, the method may further include a step of tracking a third sensor on the patellar implant during flexion and extension of the knee to track the patellar implant during the flexion and extension motion and generating a third patellofemoral joint profile from the tracked patellar implant.

Continuing in accordance with this aspect, the method may further include a step of comparing the first and third patellofemoral joint profiles.

Continuing in accordance with this aspect, the method may further include a step of selecting and placing a second patellar implant if a difference between the first and third patellofemoral joint profiles exceeds a predetermined threshold.

### BRIEF DESCRIPTION OF THE DRAWINGS

A more complete appreciation of the subject matter of the present invention and the various advantages thereof can be realized by reference to the following detailed description, in which reference is made to the following accompanying drawings:

FIG. 1 is a schematic drawing of a surgical system tracking a bone in accordance with an embodiment of the present disclosure;

FIG. 2 is a schematic drawing of the surgical system of FIG. 1 tracking a trial implant in accordance with an embodiment of the present disclosure;

FIG. 3 is a schematic drawing of the surgical system of FIG. 1 tracking an implant in accordance with an embodiment of the present disclosure;

FIG. 4 is a schematic drawing of a surgical system in accordance with an embodiment of the present disclosure, and

FIG. 5 is a flowchart showing the steps for balancing a joint utilizing a surgical system in accordance with an embodiment of the present disclosure.

### DETAILED DESCRIPTION

Reference will now be made in detail to the various embodiments of the present disclosure illustrated in the accompanying drawings. Wherever possible, the same or like reference numbers will be used throughout the drawings to refer to the same or like features within a different series of numbers (e.g., 100-series, 200-series, etc.). It should be noted that the drawings are in simplified form and are not drawn to precise scale. Additionally, the term “a,” as used in the specification, means “at least one.” The terminology includes the words above specifically mentioned, derivatives thereof, and words of similar import.

As used herein, the terms “load” and “force” will be used interchangeably and as such, unless otherwise stated, the explicit use of either term is inclusive of the other term. Similarly, the terms “implant,” and “prosthesis” will be used interchangeably and as such, unless otherwise stated, the explicit use of either term is inclusive of the other term. The term “joint implant” means a joint implant system comprising two or more implants.

In describing preferred embodiments of the disclosure, reference will be made to directional nomenclature used in describing the human body. It is noted that this nomenclature is used only for convenience and that it is not intended to be limiting with respect to the scope of the present disclosure. As used herein, when referring to bones or other parts of the body, the term “anterior” means toward the front part of the body or the face, and the term “posterior” means toward the back of the body. The term “medial” means toward the midline of the body, and the term “lateral” means away from the midline of the body. The term “superior” means closer to the head, and the term “inferior” means more distant from the head.

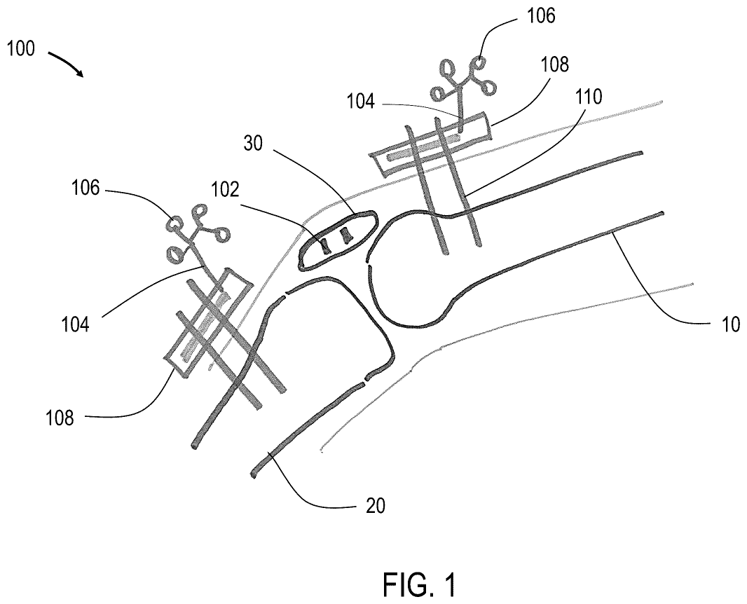

FIG. 1 shows a surgical system 100 for tracking an unresected patella 30 according to an embodiment of the present disclosure. System 100 includes one or more markers 102 attached to unresected patella 30. The markers can be attached preoperatively, intraoperatively, or postoperatively to the unresected patella. For example, markers 102 can be attached intraoperatively after a preliminary bone preparation procedure of the unresected patella. Markers 102 can be attached to the unresected patella by a temporary adhesive or other securement device. Attachment can be achieved through a medical-grade glue or gel specifically formulated to bond with both the marker and bone tissue without compromising sterility or causing adverse tissue reactions. Alternatively, temporary yet sturdy fixation members such as surgical pins, which can be delicately inserted into the bone to hold the markers in place without causing significant disruption to the surrounding tissue, small screws, designed for temporary bone fixation and easy removal post-procedure, clamps or clips, which can grip the bone's surface securely and can be repositioned as needed during the surgery, or wires, which can wrap around the patella or be threaded through small pre-drilled holes to create a secure framework for the markers.

Alternatively, markers 102 can be attached preoperatively by securing them to the unresected patella via a wearable band such as an elastic strap that wraps around the knee, an adjustable neoprene brace for added stability, or a medical-grade adhesive band that adheres to the skin. Other options could involve the use of non-invasive magnetic attachments, or Velcro straps for ease of adjustability and quick release. These attachment methods provide a temporary and stable way to position the markers accurately before surgery while ensuring they do not interfere with the knee range of motion.

Markers 102 can be magnets or other types of sensors that can be detected by a sensing device. For example, FIG. 1 shows unresected patella 30 with magnets 102 that are detected by trackers 104 positioned on a femur 10 and a tibia 20 of the patient's patellofemoral joint. Trackers 104 can be secured to femur 10 and tibia 20 via an attachment device such as a pin 110. Trackers 104 include fiducial markers 106 with a known geometry that can be tracked by a tracking device of system 100 (not shown) to measure and record the patient's knee flexion-extension. A receiver 108 configured to track the location of markers 102 is located in tracker 104. Receiver 108 can detect magnetic fields generated by markers 102, allowing it to track the position and movement of the unresected patella based on any changes of the magnetic field cause by movement of the patella relative to one or more receivers. In other embodiments, the markers can be of a different type, such as RF ID tags, optical markers, or ultrasound beacons, etc. The receiver can be tailored to detect the specific type of signal associated with these markers, such as radiofrequency signals, light patterns, or ultrasound waves, etc. Receiver 108 can be calibrated to precisely detect and interpret the signals from markers 102, enabling it to accurately identify the position and movement of the unresected patella.

System 100 can register unresected patella 30, femur 10 and tibia 20 with a probe for example. Markers 102 are secured to the unresected patella to ensure that they remain firmly and stably located on the patella during knee flexion and extension of the patient's knee without impacting the patient's range of knee motion. This ensures that proper tracking of unresected patella 30 in six degrees is collected and analyzed to identify native kinematics of patient's patellofemoral joint. System 100 can monitor the unresected patella's movement across six degrees of freedom, which includes three rotational movements and three translational movements (anterior-posterior, medial-lateral, and superior-inferior). The patella moves in multiple planes—it not only glides up and down in the trochlear groove on the femur as the knee bends and straightens but also tilts and rotates, which are critical to the knee's biomechanical function. Thus, system 100 can intraoperatively track the patellofemoral kinematics of a patient's native knee to aid with surgical decisions and optimize patella and femoral component positioning and balance. Accurate tracking of these movements by system 100 allows for a complete and precise analysis of the knee's native mechanical behavior by generating a patellofemoral joint profile which can include three-dimensional movement analysis, contract pressure and area, stress and strain patterns, velocity and acceleration, range of motion, muscle and ligament activation patterns, etc.

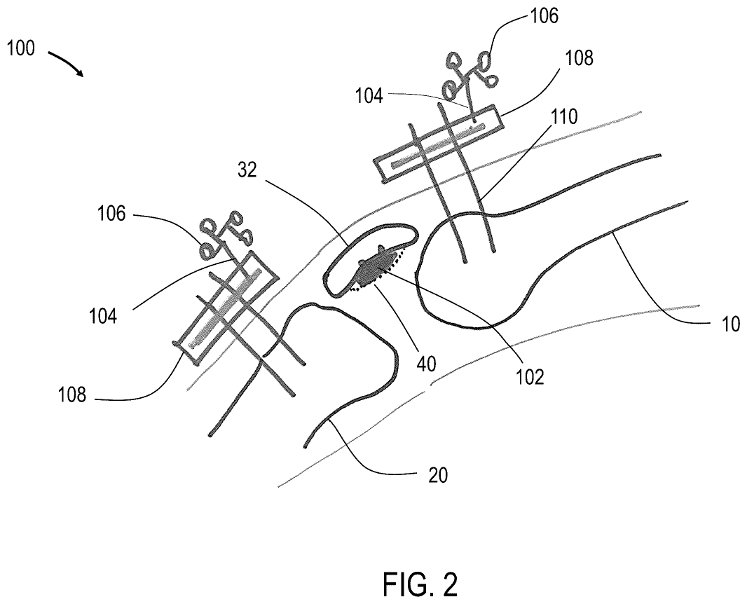

Referring now to FIG. 2, there is shown a schematic drawing of surgical system 100 tracking a patellar trial 40 in an embodiment of the present disclosure. Markers 102 can be attached to patellar trial 40 as shown in FIG. 2 or to resected patella 32. Markers 102 can be attached to patellar trial 40 as described above or these markers can be encased within the patellar trial. The selection process for the optimal size and precise placement of patellar trial 40 can be derived from a detailed analysis of the previously recorded native patellofemoral joint kinematics, as extensively outlined, and discussed with reference to FIG. 1, thereby ensuring tailored customization to the patient's native anatomical structure. Similarly, the parameters guiding the patellar resection—namely, the depth, angle, and contour—can be based on an in-depth understanding of the native patellofemoral kinematics data. Furthermore, as discussed above, markers 102 can be securely positioned externally on the patellar trial 40 or seamlessly integrated and encased within the structure of the patellar trial itself, thereby potentially enhancing the fidelity of the tracking data while minimizing the external profile of the device in the operative environment.

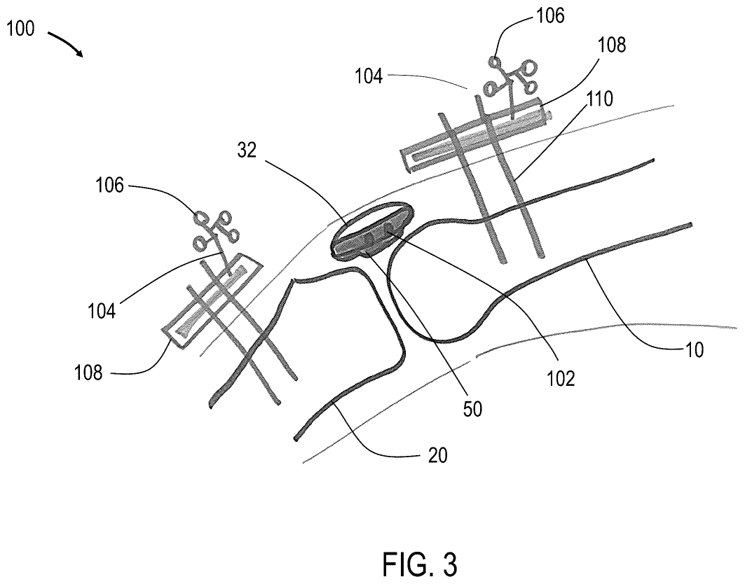

FIG. 3 shows a schematic diagram of system 100 used to track a patellar implant 50 according to another embodiment of the present disclosure. Markers 102 can be affixed to the patellar implant 50 as shown in FIG. 3, or these markers may alternatively be connected to resected patella 32. The criteria for the appropriate dimensions and exact positioning of the patellar implant 50 can be derived from a thorough assessment of patellofemoral kinematics recorded and analyzed as described above with reference to FIG. 2. This process ensures a highly personalized fit and function for the patellar implant, seamlessly corresponding to the patient's native joint movements. Markers 102 can be externally attached to the patellar implant 50 or fully encapsulated within the body of the patellar implant itself.

It should be understood that a diverse array of supplementary sensing devices may augment or, in certain instances, replace marker 102 to enhance the fidelity of the patellofemoral joint data collection. Examples of other sensors can include a pressure sensor, an inertial measurement unit (IMU), a gyroscope, an accelerometer, a force sensor, etc., each offering its own unique capabilities in monitoring various aspects of the patellofemoral joint. As an illustration of the potential for such enhancements, a pressure or force sensor might be employed in tandem with marker 102, for capturing accurate force or load measurements that occur during the patient's knee undergoing a range of flexion-extension motions. These force or load metrics provide real-time quantitative feedback which, when analyzed alongside the kinematic profile registered by markers 102 or equivalent sensors, yields a comprehensive biomechanical mapping of the patient's inherent patellofemoral kinematics. The concurrent analyzation of pressure, force, and kinematic data enables system 100 to establish a comprehensive understanding of the knee's functional patterns, including identifying any potential balancing issues—undue pressure exerted by the patella against the femur. Through this enriched data set, the selection and fine-tuning of the patellar implant can be refined with a view to achieving a more natural knee movement and ensuring that the implant's performance is harmonized with the observed native conditions to provide optimal functionality within the patient's joint structure.

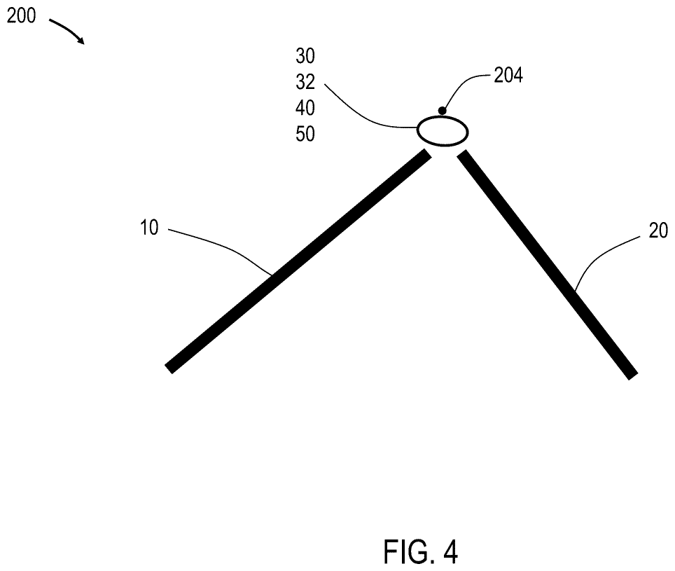

In another embodiment of the present disclosure, a surgical system 200 for tracking the kinematic behavior of various states of the patella—spanning the unresected patella 30, the patellar implant 50, and the patellar trial 40 is shown in FIG. 4. Surgical system 200 includes a sensor 204 that can be placed on any of the unresected patella 30, resected patella 32, patellar trial 40 or patellar implant 50 to track real-time monitoring of the patellofemoral kinematics of a patient's knee. Similar to system 100, system 200 can be used to track unresected patella 30 to identify the patient's native patellofemoral kinematics and joint balance. These insights lay the groundwork for informed surgical decisions regarding the sizing and precise placement of the patellar trial 40. System 200 can then be used in confirming whether the patellar trial 40 reproduces the desired patellofemoral kinematics and maintains optimal joint balance. Upon achieving a satisfactory trial fit, system 200 further guides the surgeon in validating that the chosen patellar implant 50 restores or enhances the patient's patellofemoral kinematics to the targeted functional state. System 200 eradicates the necessity for supplementary tracking devices on the tibia or femur such as trackers 104 of system 100. Sensor 204 alone is capable of precisely determining the spatial orientation and movement of the patella across all six degrees of freedom—three translational (anterior/posterior, medial/lateral, superior/inferior) and three rotational (flexion/extension, varus/valgus, internal/external rotation). This reduces the complexity and potential for error in the synchronization of multiple tracking devices but also fosters a more streamlined and less invasive surgical setup. The self-contained nature of sensor 204 enables a seamless data collection process, which effectively expedites the workflow and enhances the overall efficiency of system 200. Furthermore, by minimizing the number of instruments in the surgical environment, this embodiment of system 200 mitigates potential disruption to the surgery, thereby optimizing the conditions for successful patellar tracking and alignment.

In one embodiment of system 200, sensor 204 can include one or more inertial measurement units (IMU. IMU 204 can record the patella's disposition in relation to the femur. IMU 204 allows for accurate assessment of the patellar movement by either affixing it directly onto the patella, patellar trial or patellar implant or positioning it proximate to the patella. This strategic placement allows for a provisional epicondylar axis-a landmark reference that enables the precise determination of the native patellofemoral tracking by analyzing the relative motion and geographical relationship of the patella with the femur during the patient's knee movement. IMU 204 can include accelerometers, gyroscopes, magnetometers, etc., which work in concert to capture a full spectrum of motion data.

Alternatively, system 200 can be configured to interface with a variety of other comparable sensors that share the same functional objective. These alternative sensors, each with their unique mechanisms and specifications, are also capable of providing the necessary kinematic information essential for reestablishing the functional biomechanics of the knee joint. Regardless of the specific type of sensor deployed, the versatility of system 200 ensures seamless integration, thus enabling a tailored approach to achieving the personalized fit and motion synergy that is imperative for the optimally balanced patellofemoral joint post-surgery. Examples of other sensors can include optical tracking sensors, electromagnetic sensors, Hall effect sensors, capacitive sensors, etc.

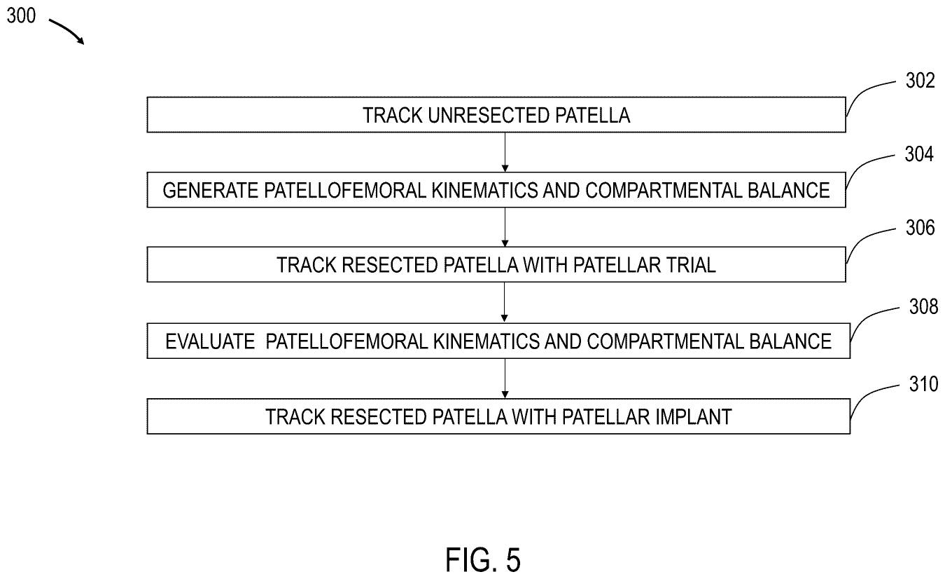

A method 300 for patellofemoral tracking and balancing using any of the surgical systems described above is shown in FIG. 5 in accordance with another embodiment of the present disclosure. In an initial step 302, the surgical system is used to track and generate the kinematic behavior of a patella prior to resection. Precise observation of the patella's movements is recorded against the relative positioning of both the femur and tibia, across a full extension-flexion range of motion that the patient's knee undergoes. Capturing this dynamic ensures a comprehensive recording to generate the patella's velocities, accelerations, and positional data in three-dimensional space, effectively encompassing all six degrees of freedom—three translational and three rotational in a step 304. Once the native patellofemoral kinematics is identified in step 304, the surgeon proceeds to step 306, wherein the native patellofemoral kinematics serve as reference for the selection and fitting of an appropriately sized patellar trial.

The selected patellar trial piece distinctly aims to replicate the patient's inherent kinematic patellofemoral characteristics, as previously articulated in step 302. Application of the corresponding sensor to the resected patella or the patellar trial, now affixed to the resurfaced patella, provides continued visibility into the patella's kinematics, allowing the surgeon to assess if the installed patellar trial maintains the natural balance and function of the patellofemoral compartment in a step 306. In an alternative embodiment, a predefined target patellofemoral kinematic range can be used as the benchmark for the selection of the patellar trial, resection, and subsequent patellar implant selection, rather than exclusively relying on replicating the patient's native patellofemoral kinematics. This target range can be derived from an extensive dataset of desired kinematic patterns identified through clinical research which may represent the optimal functional movement characteristics for a healthy knee joint.

If the patellar trial successfully mirrors the native or target patellofemoral kinematics, the process advances to a step 310, where a patellar implant is chosen based on the trial's configuration and subsequent tracking data. Appropriate sensors are once again utilized to confirm whether the installed implant indeed sustains the desired patellofemoral kinematics.

It should be noted that steps 306 and 310 can involve an iterative refinement process. This allows for precise adjustments, either minor or significant, to the trial and ultimately the implant positioning, ensuring that the kinematics and compartmental balance are optimized. Through this iterative approach, the surgical system allows a surgeon to restore the patient's patellofemoral mechanics to their preoperative state, minimizing the risk of post-surgical complications, and enhancing the potential for a swift and effective recovery.

Although the present disclosure focuses on a patellofemoral joint, it should be understood that the system and methods described herein are applicable to any joint, including but not limited to the hip joint, ankle joint, shoulder, etc. For instance, this system can be seamlessly implemented in procedures involving the hip joint, where precision and stability are paramount for the success of total hip arthroplasty or resurfacing. Similarly, in the intricate anatomy of the ankle joint, the system's advanced tracking can enhance the outcomes of ankle replacement or ligament reconstruction by providing detailed analytics of joint movement. Each adaptation to a specific joint can be based on the unique anatomical and biomechanical complexities, alongside the patient's own physiological variance, to achieve optimal surgical planning and implant positioning tailored for joint integrity and natural kinematics post-surgery.

Furthermore, although the invention disclosed herein has been described with reference to particular features, it is to be understood that these features are merely illustrative of the principles and applications of the present invention. It is therefore to be understood that numerous modifications, including changes in the sizes of the various features described herein, may be made to the illustrative embodiments and that other arrangements may be devised without departing from the spirit and scope of the present invention. In this regard, the present invention encompasses numerous additional features in addition to those specific features set forth in the paragraphs below. Moreover, the foregoing disclosure should be taken by way of illustration rather than by way of limitation as the present invention is defined in the examples of the numbered paragraphs, which describe features in accordance with various embodiments of the invention, set forth in the paragraphs below.

1. A method for patellofemoral joint balancing comprising:
positioning a first marker on an unresected patella;
tracking the first marker during flexion and extension motion of a knee to track translation and rotation of the unresected patella during the flexion and extension motion;
generating a first patellofemoral joint profile from the translation and rotation of the unresected patella;
resecting the patella based on the first patellofemoral joint profile;
selecting and placing a patellar trial on the resected patella based on the first patellofemoral joint profile;
tracking a second marker on the patellar trial or the resected patella during flexion and extension motion of the knee to track translation and rotation of the resected patella and the patellar trial during the flexion and extension motion;
generating a second patellofemoral joint profile from the translation and rotation of the resected patella and the patellar trial, and
selecting a patellar implant based on comparing the first and second patellofemoral joint profiles.

2. The method of claim 1, further including a step of selecting and placing a second patellar trial if a difference between the first and second patellofemoral joint profiles exceeds a predetermined threshold.

3. The method of claim 1, wherein the step of selecting the patellar implant includes selecting a patellar implant that is similar to the patellar trial if a difference between the first and second patellofemoral joint profiles is within a predetermined threshold.

4. The method of claim 1, further including a step of tracking a third marker on the patellar implant or the resected patella during flexion and extension of the knee to track translation and rotation of the resected patella and the patellar implant during the flexion and extension motion and generating a third patellofemoral joint profile from the translation and rotation of the resected patella and the patellar implant.

5. The method of claim 4, further including a step of comparing the first and third patellofemoral joint profiles.

6. The method of claim 5, further including a step of selecting and placing a second patellar implant if a difference between the first and third patellofemoral joint profiles exceeds a predetermined threshold.

7. The method of claim 1, further including placing a first tracker on a femur and a second tracker on a tibia, the first and second trackers including detectors to detect a position of the first and second markers.

8. The method of claim 1, wherein the step of tracking the first marker includes tracking anterior-posterior translation, medial-lateral translation, and super-inferior translation of the unresected patella.

9. The method of claim 8, wherein the step of tracking the first marker includes tracking medial rotation, lateral rotation, superior rotation, inferior rotation and axial rotation of the unresected patella.

10. The method of claim 1, wherein the step of tracking the second marker includes tracking anterior-posterior translation, medial-lateral translation, and super-inferior translation of the resected patella or the patellar trial.

11. The method of claim 10, wherein the step of tracking the second marker includes tracking medial rotation, lateral rotation, superior rotation, inferior rotation and axial rotation of the resected patella or the patellar trial.

12. The method of claim 1, wherein the step of positioning a first marker includes placing at least one magnet in contact with the unresected patella.

13. A method for patellofemoral joint balancing comprising:
positioning a first sensor on an unresected patella;
tracking the first sensor during flexion and extension motion of a knee to track the unresected patella during the flexion and extension motion;
generating a first patellofemoral joint profile from the tracked unresected patella;
resecting the patella based on the first patellofemoral joint profile;
selecting and placing a patellar trial on the resected patella based on the first patellofemoral joint profile;
tracking a second sensor on the patellar trial during flexion and extension motion of the knee to track the patellar trial during the flexion and extension motion;
generating a second patellofemoral joint profile from the tracked patellar trial, and
selecting a patellar implant based on comparing the first and second patellofemoral joint profiles.

14. The method of claim 13, wherein the step of tracking the first sensor includes tracking an inertial measurement unit on the unresected patella.

15. The method of claim 14, wherein the step of tracking the first sensor includes tracking a load sensor on the unresected patella.

16. The method of claim 15, wherein the step of tracking the first sensor includes tracking translation and rotation of the unresected patella.

17. The method of claim 16, wherein the step of tracking the first sensor includes tracking load values of the unresected patella.

18. The method of claim 17, wherein the step of tracking the first sensor includes tracking velocity and acceleration of the unresected patella.

19. The method of claim 13, wherein the step of tracking the second sensor includes tracking an inertial measurement unit on the patellar trial.

20. The method of claim 19, wherein the step of tracking the second sensor includes tracking a load sensor on the unresected patella.
